(19) United States
(12) Patent Application Publication
(10) Pub. No.: US 2024/0132425 A1
(43) Pub. Date: Apr. 25, 2024

(54) DEPOLYMERIZATION CATALYST SYSTEM AND PROCESS

(71) Applicant: Basell Poliolefine Italia S.r.l., Milan (IT)

(72) Inventors: Sandor Nagy, Seabrook, TX (US); Christopher D. Smith, Kingwood, TX (US); Daniel F. White, Houston, TX (US); Diego Brita, Ferrara (IT); Simona Guidotti, Ferrara (IT); Dario Liguori, Ferrara (IT); Francesco Menichelli, Ferrara (IT); Paolo Ruzzi, Ferrara (IT)

(73) Assignee: Basell Poliolefine Italia S.r.l., Milan (IT)

(21) Appl. No.: 18/493,300

(22) Filed: Oct. 23, 2023

Related U.S. Application Data
(60) Provisional application No. 63/380,689, filed on Oct. 24, 2022.

(51) Int. Cl. C07C 4/22; B01J 21/16; B01J 21/18; B01J 23/02; B01J 29/40; B01J 29/70

(52) U.S. Cl. CPC C07C 4/22; B01J 21/16; B01J 21/18; B01J 23/02; B01J 29/40; B01J 29/7007; C07C 2529/40

(57) ABSTRACT

Catalytic compositions and processes for depolymerizing polyolefin-based feed into useful petrochemical products are described. The compositions are a composite of a zeolite catalyst component and a co-catalyst comprising an activated clay component and/or a solid base component. These catalyst systems, along with heat, are used to both increase the depolymerization reaction rate of the feed streams and suppress poisoning effects of impurities that may be present in the polyolefin-based feed. This results in a shorter residence time in the depolymerization unit and more efficient process.

### CROSS-REFERENCE TO RELATED APPLICATIONS

The application claims the benefit of priority to U.S. Provisional Patent Application No. 63/380,689, filed on Oct. 24, 2022, which is incorporated herein by reference in its entirety.

### FIELD OF THE INVENTION

The disclosure relates to methods of depolymerizing polyolefin-based material using a catalyst system comprising a combination of a zeolite catalyst and a co-catalyst comprising an activated clay component and/or a solid base component to form useful petrochemical products, such as olefin monomers.

### BACKGROUND OF THE INVENTION

Heightened standards of living and increased urbanization have led to an increased demand for polymer products, particularly polyolefin plastics. Polyolefins have been frequently used in commercial plastics applications because of their outstanding performance and cost characteristics. Polyethylene (PE), for example, has become one of the most widely used and recognized polyolefins because it is strong, extremely tough, and very durable. This allows for it to be highly engineered for a variety of applications. Similarly, polypropylene (PP) is mechanically rugged yet flexible, is heat resistant, and is resistant to many chemical solvents like bases and acids. Thus, it is ideal for various end-use industries, mainly for packaging and labeling, textiles, plastic parts, and reusable containers of various types.

The downside to the demand for polyolefin plastics is the increase in waste. Post-consumer plastic waste typically ends up in landfills, with about 12% being incinerated and about 9% being diverted to recycling. In landfills, most plastics do not degrade quickly, becoming a major source of waste that overburdens the landfill. Incineration is also not an ideal solution to treating the plastic wastes as incineration leads to the formation of carbon dioxide and other greenhouse gas emissions. As such, there has been much interest in developing methods of recycling plastic waste to reduce the burden on landfills while being environmentally friendly.

A drawback to the recycling of plastic wastes is the difficulty in successfully producing commercially usable or desirable products. Plastic waste recycling currently includes washing the material and mechanically reprocessing it; however, the resulting pellets remain contaminated with impurities such as food residue, dyes, and perfume. These impurities render the pellets undesirable for many uses based on both performance and appearance. Further, it is difficult to obtain a pure stream of any particular polymer, resulting in a mixed plastic waste stream that may not have the desired properties post-recycling.

Recent advances have focused on converting plastic waste to useable products like fuel sources or commercially important raw materials. Methods of performing pyrolysis of the plastic waste stream followed by catalytic depolymerization have been developed to generate various products: gases, gasoline fractions, kerosene fractions, diesel fractions, and waxes. Unfortunately, the catalysts themselves tend to be easily poisoned by other chemicals in the polyolefin waste feed, resulting in processes that are costly and time-consuming because they require significant energy to fully decompose polyolefin wastes to useful classes of products.

Despite the advances made in recycling polyolefins, there is a continued need for the development of a robust process for the conversion of polyolefin-rich waste feeds to useful petrochemical products. Ideally, these processes will overcome ‘poisoning’ from other polymers and contaminants that may be present in the waste feed.

### SUMMARY OF THE INVENTION

The present disclosure provides novel compositions and methods for thermally depolymerizing polyolefin-based material in the absence of oxygen. The presently disclosed compositions are combinations of a zeolite catalyst component and a co-catalyst comprising an activated clay component and/or a solid base component to form useful petrochemical products, such as olefin monomers. The combinations of the zeolite catalyst component and the co-catalyst have a synergistic effect for increasing the rate of the depolymerization reactions while also suppressing any poisoning effects from impurities that may be present in the teed stream, or from degradation products of the impurities. Specifically, the zeolite catalyst component and the co-catalyst, comprising an activated clay component and/or a solid base component, f=orm a robust depolymerization catalyst system. Components of the depolymerization catalyst system are added to separate depolymerization reaction zones in a depolymerization process comprising at least two depolymerization reaction zones. The reaction mixture in each pyrolysis reaction zone is heated in the absence of oxygen in a process called thermolysis to quickly generate useful petrochemical products.

In some embodiments, a catalyst system for depolymerizing polymers comprises a zeolite catalyst component and a co-catalyst, comprising an activated clay component, a solid base component, or a combination thereof.

In some embodiments, a process for depolymerizing polymers comprises: a) adding a polyolefin-based feed stream and a first co-catalyst to a first pyrolysis reaction zone to form a first reaction mixture, wherein the first co-catalyst comprises an activated clay component, a solid base component, or a combination thereof; b) reacting the first reaction mixture under first depolymerization conditions in the absence of oxygen to form a first vapor product and char; c) adding the first vapor product to a first condensation zone wherein the first vapor product is subjected to condensing conditions to form a second vapor product and a first liquid product; d) adding the first liquid product and a zeolite catalyst component to a second pyrolysis reaction zone to form a second reaction mixture; and e) reacting the second reaction mixture under second depolymerization conditions in the absence of oxygen to form a third vapor product, a second liquid product, and char, wherein the second liquid product comprises one or more olefin monomers.

In some embodiments, a depolymerization system comprises: a) a pyrolysis reaction zone to heat a mixture of a polyolefin-based feed stream, a zeolite catalyst, and a co-catalyst, comprising an activated clay component, a solid base component, or a combination thereof, to produce a vapor product and char; and b) a condensation unit to condense the vapor product to produce a second vapor product and a liquid product comprising one or more olefin monomers.

In some embodiments, a depolymerization system comprises: a) a first pyrolysis reactor to heat a mixture of a polyolefin-based feed stream a first co-catalyst to produce a first vapor product and char; b) a first condensation unit to condense the first vapor product to produce a second vapor product and a first liquid product; c) a second pyrolysis reactor to heat a mixture of the first liquid product, and optionally a second co-catalyst to produce a second liquid product and char; and d) a second condensation unit to condense the second liquid product, and optionally the second vapor product to produce a third vapor product and a third liquid product comprising one or more olefin monomers.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other catalyst compositions and/or processes for carrying out the same purposes of the present invention. It should also be realized by those skilled in the art that such equivalent constructions do not depart from the spirit and scope of the invention as set forth in the appended claims. The novel features which are believed to be characteristic of the invention, both as to its compositions and methods, together with further objects and advantages will be better understood from the following description.

### BRIEF DESCRIPTION OF THE FIGURES

The claimed subject matter may be understood by reference to the following description taken in conjunction with the accompanying drawings, in which like reference numerals identify like elements, and in which:

FIG. 1 is a simplified flow diagram of the disclosed process having two depolymerization reaction zones and two condensation zones according to embodiments of the invention;

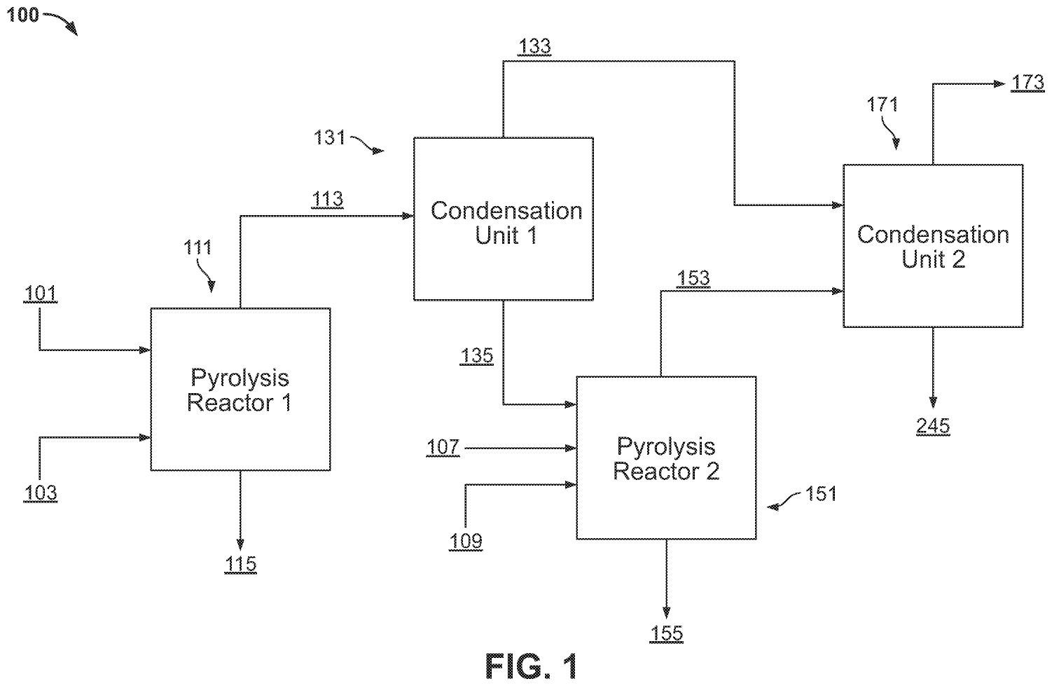

FIG. 2 is a flow diagram of certain embodiments of the disclosed process having two depolymerization reaction zones and two condensation zones; and

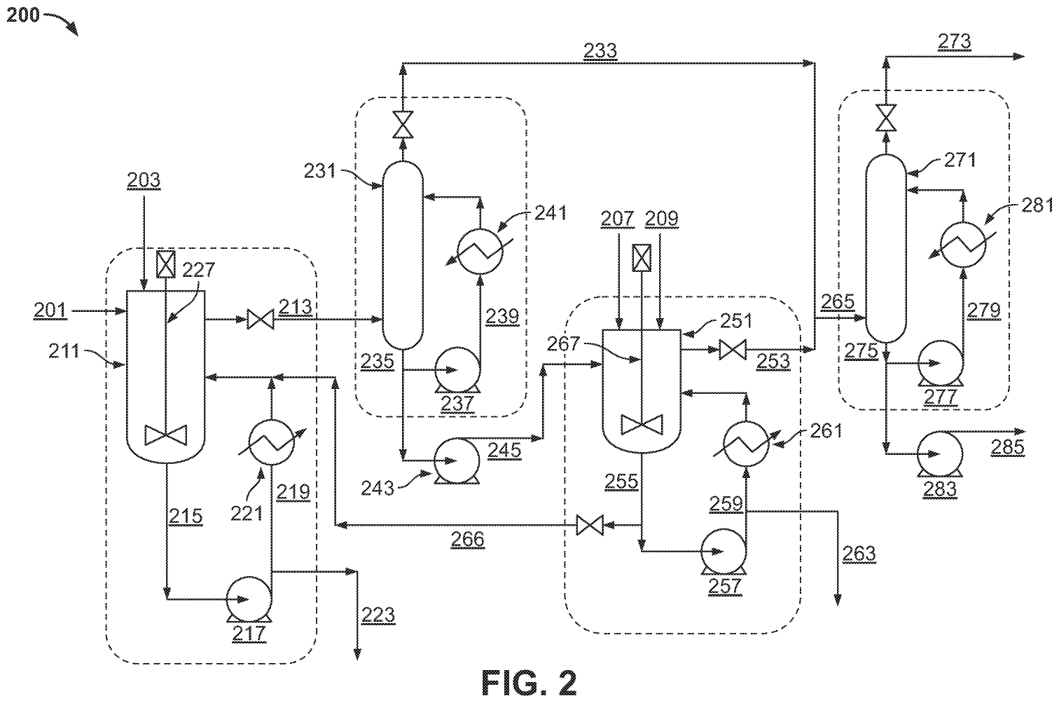

FIG. 3 shows overlaid graphs of zeolite catalyst performance alone and with addition of co-catalysts in depolymerizing samples of waste plastic.

While the disclosed process and composition are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase. It must also be noted that, as used in the specification and the appended claims, the singular forms “a,” “an,” and “the” include plural references unless otherwise specified.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “activated clay” describes clay or clay minerals, including, but not limited to, smectites or bentonites (which include montmorillonite, nontronite, beidellite and saponite); alumino-silicates, sepiolite, attapulgite (palygorskite); kaolins; and other fuller's earths which have been chemically treated with dilute acids (e.g., sulfuric acid) and thermally heat treated between 100° C. and 200° C.

As used herein, “char” refers to coke, a carbon-containing solid, that accumulates on the catalyst particles during pyrolysis.

As used herein, “feed stream” refers to a supply of polyolefin-based material for depolymerization. Depending on the depolymerization unit, the feed stream can be a continuous supply of material or a batch of material. The feed stream can be pure polyolefins or can be a mix of polyolefins with non-polyolefin components.

As used herein, “non-polyolefin components” refers to material present in a polyolefin-based feed, or waste, stream that can reduce the abilities of a zeolite to catalyze the depolymerization of the polyolefins that are present in the stream. Examples of non-polyolefin components include non-polyolefinic polymers with high oxygen and/or nitrogen content.

As used herein, “post-consumer waste” refers to a type of waste produced by the end consumer of a material stream.

As used herein, “post-industrial waste” refers to a type of waste produced during the production process of a product.

As used herein, “reaction zone” refers to a chamber sufficiently enclosed to maintain selected operating conditions within the chamber to produce a desired reaction, such as a pyrolysis reaction zone or a condensing reaction zone. In some embodiments, each reaction zone can be a separate reactor. In some embodiments, a single vessel can contain a plurality of reaction zones.

As used herein, “residence time” refers to the time needed to depolymerize a batch of polymer waste in a depolymerization unit.

As used herein, “thermolysis” refers to a thermal depolymerization reaction occurring in the absence of oxygen.

As used herein, “waste stream” is a type of feed stream comprising material that has been discarded as no longer useful, including but not limited to, post-consumer and post-industrial waste.

As used herein, “zeolite” refers to an aluminosilicate mineral with a microporous structure. Zeolites are, in one aspect, useful as catalysts for the processes disclosed herein. Zeolites can occur naturally or can be produced industrially.

As used herein, the terms “depolymerization half time” or “half time of depolymerization” refer to the time needed to achieve a 50% loss of mass of a sample at a specific temperature during a TGA thermolysis reactions. The depolymerization half time is related to the residence time that would be needed for large scale industrial depolymerization reactors.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the disclosure.

The term “pure” as used in reference to the feed stream refers to a feed that is 100% polyolefin, but does not mean that the feed contains only one type of polyolefin. Rather, a “pure” feed stream can have a mixture of polyolefins such as low-density polyethylene, high density polyethylene, polypropylene and combinations thereof.

The terms “polyolefin-based” and “polyolefin-rich”, in reference to materials, feed streams, or waste streams, are used interchangeable to refer to a mixture that is at least 80% polyolefin.

All concentrations herein are by weight percent (“wt. %”) unless otherwise specified.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed and excludes all additional elements.

The phrase “consisting essentially of” excludes additional material elements but allows the inclusions of non-material elements that do not substantially change the nature of the invention.

The phrase “substantially all of” means greater than or equal to 95 wt %, greater than or equal to 99 wt %, greater than or equal to 99.5 wt %, or greater than or equal to 99.9 wt %.

The following abbreviations are used herein:

| ABBREVIATION | TERM |
|---|---|
| Beta | Beta (or BEA) zeolite |
| EVA | ethylene vinyl acetate |
| EVOH | ethylene vinyl alcohol |
| HDPE | High density polyethylene |
| H-USY | H-ultra-stable zeolite Y |
| LDPE | Low-density polyethylene |
| MPO | Municipal polyolefin waste |
| NPC | Non-polyolefin component |
| PE | polyethylene |
| PET | poly(ethylene terephthalate) |
| PP | polypropylene |
| sccm | standard cubic centimeter |
| | per minute @ 0° C./32° F. |
| TGA | Thermogravimetric Gravimetric Analysis |
| USY | ultra-stable zeolite Y |
| vo1% | volume percent |
| wt % | weight percent |
| ZSM-5 | Zeolite Socony Mobil-5 |

### Catalyst Composition

The present disclosure provides catalytic compositions for recycling polyolefin-based materials into commercially important raw material. In some embodiments, a catalyst composition for depolymerizing a polyolefin-based feed stream in a depolymerization unit comprises or consists essentially of a zeolite catalyst component and a co-catalyst, comprising an activated clay component and/or a solid base component. The components of the composite catalyst system work synergistically to increase the rate of depolymerization, thus reducing the amount of time the polyolefin-based feed stream spends in the depolymerization unit. The improvement in the rate of depolymerization occurs even in the presence of impurities, such as non-polyolefin components, in the feed stream that can reduce the catalytic activity of the zeolite in the absence of the co-catalyst, comprising an activated clay component and/or a solid base component.

Zeolite Catalyst Component

Zeolites are solid acid catalysts with an open, three-dimensional crystal structure with many pores and acidic active sites where chemical reactions such as the thermal depolymerization of polyolefins can take place. The depolymerization proceeds via the production of the intermediate carbenium ion by hydrogen transfer reactions initiated by the zeolites' acid sites and subsequent unzipping. Essentially, zeolites rely on strongly acidic sites to crack the polyolefins. This cracking process begins on the surface of the zeolite, because the polymer needs to be broken into smaller molecules before entering the internal voids of these solids, due to the small size of their openings. This leads to more contact between the polyolefin and the catalyst, resulting in a faster rate of depolymerization and a shorter residence time in the depolymerization unit.

Zeolites have many advantages for depolymerizing polyolefins, including the ability to adjust the acidity and void sizes to address specific feed stream characteristics. Additionally, zeolites are heterogeneous catalyst, allowing for easy separation and reusability from the depolymerization products or by-products.

Zeolites catalyst, however, are susceptible to poisoning and reduced catalytic activity in the presence of non-polyolefin components such as polymers with nitrogen-containing groups and/or high oxygen content, and/or their depolymerization products such as furfural. Additionally, many of the substrates with high oxygen and nitrogen content can form coke deposits on zeolites, further reducing the zeolites' activity. Feed streams of polyolefin wastes are rarely pure, even after multiple separation steps in recycling facilities, and a small addition of a non-polyolefin component can suppress the zeolites' catalytic abilities and decrease its depolymerization rate by as much as 83%.

Any zeolite catalyst that is capable of catalyzing the depolymerization reaction of polyolefins can be used in the composite catalyst. In some embodiments, the composite catalyst includes commercially available zeolites including but not limited to, beta zeolite (beta), Zeolite Socony Mobil-5 (ZSM-5), zeolite Y (Y), ultra-stable zeolite Y (USY), amorphous acidic AlSiOₓ such as Siral® 40, or combinations thereof. Combinations of zeolites may be useful to address specific polyolefin-based feed content or can be used to offset costs associated with using only an expensive zeolite in the composite.

In some embodiments, the zeolite catalyst component has a SiO₂/Al₂O₃ mole ratio of less than or equal to 200, less than or equal to 100, less than or equal to 50, less than or equal to 25, or less than or equal to 15. In some embodiments, the zeolite catalyst component has a SiO₂/Al₂O₃ mole ratio in the range of from 200:1 to 3:1, from 150:1 to 3:1, from 100:1 to 3:1, from 50:1 to 3:1, from 25:1 to 3:1, or from 15:1 to 3:1. In some embodiments, the zeolite catalyst component has a SiO₂/Al₂O₃ mole ratio in the range of from 200:1 to 5:1, from 150:1 to 5:1, from 100:1 to 5:1, from 50:1 to 5:1, from 25:1 to 5:1, or from 15:1 to 5:1. In some embodiments, the zeolite catalyst has a SiO₂/Al₂O₃ mole ratio in the range of from 200:1 to 10:1, from 150:1 to 10:1, from 100:1 to 10:1, from 50:1 to 10:1, from 25:1 to 10:1, or from 15:1 to 10:1. In some embodiments, the zeolite catalyst has a SiO₂/Al₂O₃ mole ratio in the range of from 200:1 to 1:1, from 150:1 to 1:1, from 100:1 to 1:1, from 50:1 to 1:1, from 25:1 to 1:1, or from 15:1 to 1:1.

Co-Catalyst

The present compositions and methods overcome these issues by combining at least one zeolite with a co-catalyst, comprising an activated clay component and/or a solid base component, that is able to suppress poisoning effects from impurities, such as non-polyolefin components, in polyolefin-based feed streams. The co-catalyst maintains and/or restores the ability the zeolite catalyst component to crack the polyolefins. In addition to reducing and/or preventing zeolite suppression by impurities in the polyolefin-rich feed, the co-catalyst, comprising an activated clay component and/or a solid base component, unexpectedly works synergistically with one or more zeolites to improve the depolymerization rate of a polyolefin-based feed stream to a greater extent than the zeolite alone, especially if the polyolefin-based feed contains impurities.

Activated Clay

For the purposes of the present invention, activated clay is understood to mean a clay that has been subjected to a thermal or chemical activation process. In some embodiments, the clay is activated by treatment with diluted sulfuric acid and drying at a temperature in the range of from 100° C. to 200° C. Activated clay is also to be understood as “acid washed clay”. Clay (without being subjected to an activation process) comprises one or more of the following nonlimiting examples of clay minerals (phyllosilicates): attapulgite (palygorskite), albite, alumino-silicates, beidellite, bentonite, chlorites, fuller's earths, gibbsite, goethite, halloysite, hectorite, hematite, alpha hematite, Mite, kaolinite, kaolins, metahalloysite, micas (including muscovite 2-M1 and/or illite-1M), montmorillonite, nontronite, pyrite, quartz, talc, saponite, sauconite, sepiolite, smectites, vermiculite, and other similar compositions. In some embodiments, the activated clay is a mixture of several activated clays with different characteristics, or an activated clay and one or more inactivated clays.

In some embodiments, the chemical composition of the activated clay has an Al₂O₃ content greater than 10 wt %, in the range of from 10 wt % to 50 wt %, or from 15 wt % to 30 wt %. In some embodiments, the activated clay has an Fe₂O₃ content greater than 5 wt %, in the range of from 1 wt % to 60 wt %, from wt % to 20 wt %. or from 5 wt % to 20 wt %. In some embodiments, the activated clay comprises other components, such as SiO₂, Na₂O, to K₂O.

The activated clay co-catalyst in the present composite catalyst is an inorganic material that has an acidic Character. Zeolites rely on strongly acidic sites to initiate the depolymerization of polyolefins. As such, acidic co-catalyst compounds can be used as co-catalyst without affecting the zeolite's initiation method.

The amount of activated clay co-catalyst(s) in the composite catalyst will depend on the content of the polyolefin feed, as well as the types of non-polyolefin components, if any, and the amount thereof in the feed stream. in some embodiments, the total amount of activated day co-catalysts is in the range of from 10 wt % to 90 wt % or from 20 wt % to 60 wt % of the composite catalyst, with the remaining balance being the total amount of zeolites and solid base, if any. Alternatively, the total amount of activated clay co-catalyst is in the range of from 40 wt % to about 75 wt % of the composite catalyst. Alternatively, the total amount of activated clay co-catalysts is in the range of from 70 wt % to 90 wt % of the composite catalyst. Alternatively, the total amount of activated clay co-catalysts is in the range of from 50 wt. %% u to 75 wt. of the composite catalyst.

Solid Base

In some embodiments, a solid base component is: a layered double hydroxide composition; an activated carbon composition; or a combination thereof.

In some embodiments, a solid base component is: a layered double hydroxide composition; an MR composition, wherein M is an alkali metal, an alkaline earth metal, a transition metal, a post-transition metal, metalloid, or a combination thereof and R is an oxide, a hydroxide, a carbonate, a sulfate, a sulfide, nitrate, nitride, phosphate, phosphite, a halide, or a combination thereof; an activated carbon composition; or a combination thereof.

In some embodiments, the layered double hydroxide is a hydrotalcite, a quiminite, a fougerite, a woodwardite, a cualstibite, a glaucocerinite, a wermlandite, a hydrocalumite, or a combination thereof. In some embodiments, the layered double hydroxide is hydrotalcite.

In some embodiments, the MR composition is Ca(OH)₂, Mg(OH)₂, Ba(OH)₂, Sr(OH)₂, CaO, Al₂O₃, or a combination thereof. In some embodiments, the MR composition is Ca(OH)₂, or a combination thereof. In some embodiments, the solid base co-catalyst is a metal oxide or metal hydroxide. Any known metal oxide or metal hydroxide can be used including those containing metals from Groups 2-8 and 11-16, as well as the lanthanoids and actinoids. Exemplary solid inorganic co-catalyst include Ca(OH)₂, Mg(OH)₂, Ba(OH)₂, Sr(OH)₂, NaOH, KOH, CaO, and Al₂O₃. Any combination of the solid base co-catalysts can be used in some embodiments, the composite catalyst has both Al₂O₃ and Ca(OH)₂

The solid base component can be a silicate, an aluminosilicate, a carbonate, phosphate, oxide or a hydroxide. Zeolites rely on strongly acidic sites to initiate the depolymerization of polyolefins. Unexpectedly, however, it was found that basic co-catalyst compounds can also be used without neutralizing the zeolite's acidic sites or affecting its ability to crack the polyolefins.

The amount of solid base co-catalysts) in the composite catalyst will depend on the content of the polyolefin feed, as well as the types of non-polyolefin components, if any, and the amount thereof in the feed stream. In some embodiments, the total amount of solid base co-catalysts is in the range of from 10 wt % to 90 wt % or from 20 wt % to 60 wt % of the composite catalyst, with the remaining balance being the total amount of zeolites and activated clay, if any. Alternatively, the total amount of solid base co-catalyst is in the range of from 40 wt % to about 75 wt % of the composite catalyst. Alternatively, the total amount of solid base co-catalysts is in the range of from 70 wt % to 90 wt % of the composite catalyst. Alternatively, the total amount of solid base co-catalysts is in the range of from 50 wt. % to 75 wt. % of the composite catalyst.

### Depolymerization Process

In some embodiments of the present disclosure, the composite catalyst is a combination of zeolite catalyst with an activated clay co-catalyst, a solid base co-catalyst, or a combination thereof, wherein the zeolite catalyst is present in the range of from 25 wt % to 50 wt % of the composite catalyst. The zeolite catalyst can be one or more ZSM-5 zeolites, one or more Beta zeolites, one or more Y zeolites, one or more ultra-stable Y zeolites, or a combination thereof. In some embodiments, the zeolite catalyst is one or more ultra-stable Y zeolites.

The presently described composite catalysts can be used to thermally degrade or depolymerize a feed stream comprising material with a single polyolefin component or a mixture of polyolefin components in any amount. Any polyolefin can be present in the feed stream, including but not limited to, polyethylene (both high and low density), polypropylene, ethylene-propylene copolymers, polybutene-1 polyisobutene, and copolymers thereof. Further, the feed stream is not limited to any particular form so films, foams, textiles or other shaped material can be treated with the described methods. The polyolefins can be obtained from waste streams, including post-consumer waste streams, post-industrial waste streams, or combinations thereof.

In some embodiments, the feed stream further comprises one or more non-polyolefin components that decrease the catalytic activity of a zeolite. Alternatively, the feed stream may further comprise one or more non-polyolefin components that generate degradation products that decrease the catalytic activity of a zeolite. While many chemicals fall into this category, non-polyolefin polymers are most likely to be present in polyolefin-based feed streams, particularly where the feed stream is a waste stream. In particular, non-polyolefin polymers with nitrogen or high oxygen content such as polyaramids, acrylates, nylons, polyurethanes, cellulose and polyvinyl polymers may be present in the feed stream. These polymers are commonly found at waste sites and are difficult to completely separate from polyolefins. Many of these polymers degrade into problematic products that are capable of reducing the zeolite's catalytic abilities, such as furfural, caprolactam, various amines, phenols, and esters. Alternatively, non-polyolefin components such as pigments containing nitrogen may be present in polyolefin-based waste stream and able to decrease the catalytic activity of a zeolite.

In some embodiments, a process for depolymerizing polymers comprises: a) adding a polyolefin-based feed stream and a first co-catalyst to a first pyrolysis reaction zone to form a first reaction mixture, wherein the first co-catalyst comprises an activated clay component, a solid base component, or a combination thereof; b) reacting the first reaction mixture under first depolymerization conditions in the absence of oxygen to form a first vapor product and a first liquid product comprising char; c) adding the first vapor product to a first condensation zone, wherein the first vapor product is subjected to condensing conditions to form a second vapor product and a second liquid product; d) adding the second liquid product and a zeolite catalyst component to a second pyrolysis reaction zone to form a second reaction mixture; and e) reacting the second reaction mixture under second depolymerization conditions in the absence of oxygen to form a third vapor product and a third liquid product comprising char, wherein the third liquid product comprises one or more olefin monomers.

In further embodiments of the process for depolymerizing polymers, the polyolefin feed stream comprises polyethylene, polypropylene, or a combination thereof. In some embodiments, the polyolefin feed stream comprises 0 wt % up to 20 wt %, 0 wt % up to 15 wt %, 0 wt % up to 10 wt %, or 0 wt % up to 5 wt % of an impurity, wherein the weight percentage is based on the total weight of the polyolefin feed stream. In some embodiments, the impurity comprises one or more members of the group consisting of polyethylene terephthalate, polystyrene, water, chlorine, or a combination thereof.

In some embodiments, the process for depolymerizing polymers further comprises adding a second co-catalyst to the second pyrolysis reaction zone, wherein the second co-catalyst comprises an activated clay component, a solid base component, or a combination thereof, and the second co-catalyst composition is different than the first co-catalyst composition.

In some embodiments of the process for depolymerizing polymers, the first co-catalyst comprises a solid base component. In some embodiments of the process for depolymerizing polymers, the first co-catalyst comprises an activated clay component. In some embodiments of the process for depolymerizing polymers, the second co-catalyst comprises a solid base component. In some embodiments of the process for depolymerizing polymers, the second co-catalyst comprises an activated clay component. In some embodiments of the process for depolymerizing polymers, the first co-catalyst comprises a solid base component and the second co-catalyst comprises an activated clay co-catalyst. In some embodiments of the process for depolymerizing polymers, the first co-catalyst comprises an activated clay co-catalyst and the second co-catalyst comprises a solid base component.

In some embodiments of the process for depolymerizing polymers further comprises adding the second vapor product to the to the second condensation zone.

In some embodiments of the process for depolymerizing polymers, the first depolymerization conditions comprise a temperature in the range of from 250° C. to 600° C., from 400° C. to 600° C., from 425° C. to 550° C., or from 450° C. to 500° C., a pressure in the range of from 100 kPa to 1,000 kPa, from 100 kPa to 700 kPa, from 150 kPa) to 600 kPa, or from 200 kPa to 500 kPa, or a combination thereof.

In some embodiments of the process for depolymerizing polymers, the second depolymerization conditions comprise a temperature in the range of from 250° C. to 600° C., from 250° C. to 450° C., from 275° C. to 425° C., or from 300° C. to 400° C., a pressure in the range of from 100 kPa to 1,000 kPa, from 100 kPa to 700 kPa, from 150 kPa to 600 kPa, or from 200 kPa to 500 kPa, or a combination thereof.

In some embodiments of the process for depolymerizing polymers, the first condensing conditions comprise a temperature in the range of from 20° C. to 250° C., from 30° C. to 200° C., or from 40° C. to 150° C., a pressure in the range of from 100 kPa to 200 kPa, from 110 kPa to 190 kPa, or from 120 kPa to 180 kPa, or a combination thereof.

In some embodiments of the process for depolymerizing polymers, the second condensing conditions independently comprise a temperature in the range of from 20° C. to 100° C., from 30° C. to 90° C., or from 40° C. to 80° C., a pressure in the range of from 30 kPa to 200 kPa, from 50 kPa to 170 kPa, or from 70 kPa to 130 kPa, or a combination thereof.

### Certain Embodiments

In some embodiments, the polyolefin-based feed stream can be treated in batches in the depolymerization unit due to the residence time needed to fully depolymerize the feed stream. The estimated residence time for each batch will be in the range of from 30 minutes to 300 minutes, from 40 minutes to 200 minutes, or from 50 minutes to 100 minutes, depending on the design of the &polymerization system.

In some embodiments, the process is operated in a continuous mode. Waste plastic materials 201 are fed into the first depolymerization reactor 211. Steps are taken to prevent introducing oxygen-containing atmosphere into the system. The barrier to the oxygen-containing atmosphere can be obtained in different ways such as, but not limited to, a nitrogen blanketing system, a vacuum system, or a combination thereof connected to a barrel of the extruder. In some embodiments, the plastic waste mixture 201 is charged into the feeding system of the depolymerization reactor 201 by means of a hopper, or two or more hoppers in parallel, and the oxygen present in the atmosphere of the plastic waste material is substantially eliminated inside the hopper(s).

The process according to the present disclosure is very flexible and can be fed with a wide range of plastic waste composition as, for example, a heterogeneous mixture of waste plastic materials in which polyolefins are the most abundant component but for which a further sorting step is no longer economical. In some embodiments, the plastic waste mixture comprises polyethylene, polypropylene, or combination thereof, in an amount greater than or equal to 70 wt %, greater than or equal to 80 wt %, or greater than or equal to 90 wt %.

In some embodiments, the waste plastic material undergoes a pretreatment stage in which it is melted by heat and possibly mixed with an additive which can be an alkaline material. By the melting pretreatment, a non-uniform mixture of different kinds of waste plastics can be transformed into a mass of uniform plastic composite. The heating temperature in the pretreatment stage is appropriately set to a temperature in accordance with the kind and content of the plastic contained in the waste plastic material such that pyrolytic decomposition of the plastic material to be treated is inhibited. Such a temperature is, in general, within a range of 100° C. to 300° C., and preferably, 150° C. to 250° C.

At a temperature close to 300° C. or more, elimination of HCl from the PVC resin possibly present, takes place. The HCl forming gas can be either removed via a venting system and successively neutralized or trapped if the waste plastic material is mixed with an alkaline material during the melting/kneading pretreatment. For performing the melting operation, ordinary kneaders, extruders with a screw, and the like are applicable. In some embodiments, an extruder melts the plastic scrap 201 and brings it a high temperature, such as, but not limited to, 250° C. to 350° C., and injects it into the first depolymerization reactor 211. The extruder may receive the plastic scrap cut in small pieces into the feed hopper, convey the stream in the melting section and heat the polymer by combined action of mixing energy and heat supplied by barrel heaters. Any extrusion systems can be applied, as single screw extruders, twin screw extruders, twin screw extruders with gear pump, or combination of the above.

Additives can be optionally incorporated in the melt aiming at reducing corrosivity of plastic scrap received or to improve conversion process in the reaction section. During the extrusion, one or more degassing steps can be foreseen to remove residual humidity present in the product. Before being fed to the first depolymerization reactor 211, the melt stream can be filtered by mechanical means in order to remove solid impurities present in the plastic waste. Several types of melt filtration units can be applied, depending on amount and particle size of the solid impurities. In some embodiments, self-cleaning melt filters can be operated for an extended period (several days) without manual intervention to replace filtration elements.

In some embodiments, a melt filter is based on a circular perforated plate as melt filtration element, having holes produced by laser or by machining, where solids are accumulated. Accumulation of impurities may increase differential pressure across the melt filter. In order to perform the in-line cleaning of the filtration element, a rotating scraper removes the accumulated impurities and guides them to a discharge port, that is opened for short time to purge out of the process contaminated material.

This cycle can be repeated several times (up to operation time of several days) without manual intervention or need to stop production for the time needed to replace the filtration element. Another option of self-cleaning melt filter is based on the application of continuous filtering metal bands through which polymer flow is passed. Impurities are accumulated on the metal filter generating an increases of pressure. Accordingly, the clogged filtering band section is pushed out of the polymer passage area and clean section is then inserted. This process is automatic and allows to operate for long time (up to several days) without manual intervention or need to stop production for the time needed to replace the filtration element.

In some embodiments, a process for depolymerizing 200 waste plastic material and producing a pyrolytic product comprises feeding a polyolefin-rich waste feed 201 and a first co-catalyst composition 203, in an oxygen-free atmosphere, into a feeding system comprising a screw extruder, mixer, or other means for heating the mixture to the melting temperature of the plastic material to produce a molten plastic feed stream 201. The first co-catalyst composition 203 comprises a solid base, an activated clay, or a combination thereof.

In some embodiments, the first co-catalyst composition 203 comprises an activated clay component which is one or more members selected from the group consisting of montmorillonite, sauconite, nontronite, hectorite, beidellite, saponite, bentonite, or a combination thereof, or is bentonite comprising Na-montmorillonite, Ca-montmorillonite, or a combination thereof.

In some embodiments, the first co-catalyst composition 203 comprises a solid base component is one or more members selected from the group consisting of: a) a layered double hydroxide composition; and b) an activated carbon composition. In some embodiments, the first co-catalyst composition 203 comprises a solid base component is one or more members selected from the group consisting of: a) a layered double hydroxide composition; b) an MR composition, wherein M is an alkali metal, an alkaline earth metal, a transition metal, a post-transition metal, metalloid, or a combination thereof, and R is an oxide, a hydroxide, a carbonate, a sulfate, a sulfide, nitrate, nitride, phosphate, phosphite, a halide, or a combination thereof; and c) an activated carbon composition. In some embodiments, the layered double hydroxide is a hydrotalcite, a quintinite, a fougerite, a woodwardite, a cualstibite, a glaucocerinite, a wermlandite, a hydrocalumite, or a combination thereof, or is hydrotalcite. In some embodiments, the MR composition is Ca(OH)₂, Mg(OH)₂, Ba(OH)₂, Sr(OH)₂, CaO, Al₂O₃, or a combination thereof, or is Ca(OH)₂, or a combination thereof.

The amount of first co-catalyst 203 used in the present methods can be limited by the requirements of the depolymerization unit. The first co-catalyst 203 is a solid that contributes to the dead volume in the unit during the depolymerization reaction. The lower the dead volume, the more polymer that can be depolymerized. In some depolymerization units, the amount of first co-catalyst is in the range of from 0.1 wt % to 20 wt %, from 0.2 wt % to 15 wt %, from 0.5 wt % to 10 wt %, or from 1.0 wt % to 8 wt %, wherein weight percentages are based on the total weight of waste plastic 201 and first co-catalyst component 203 fed to the first depolymerization reactor 211.

Care should be taken for not introducing oxygen containing atmosphere into the system. The barrier to the potentially oxygen-containing atmosphere can be obtained in different ways such as nitrogen blanketing or vacuum system connected to a barrel of the extruder. The molten plastic feed stream is fed to a first depolymerization reactor 211, which is a continuously stirred tank reactor maintained at a temperature ranging from 250° C. to 600° C., from 400° C. to 600° C., from 425° C. to 550° C., or from 450° C. to 500° C., and operated under a pressure ranging from 100 kPa to 1,000 kPa, from 100 kPa to 700 kPa, from 150 kPa to 600 kPa, or from 200 kPa to 500 kPa, in which depolymerization takes place to form a first gaseous effluent and a first liquid effluent.

In some embodiments, the first depolymerization reactor 211 has a cylindrical section with a rounded bottom with a mixer 227 installed in the vertical axis of the reactor 211, completed with a gear motor which allows the blades of the mixer rotating in order to maintain the system in stirred state. The design of the mixer and the power of the motor can vary in respect of the reactor content, volume and shape, however, as a non-limiting example, mixer 227 operates with a power input in the range of from 0.2 kW/m 3 to 4 kW/m³, from 0.25 kW/m 3 to 2 kW/m³, or from 0.3 kW/m 3 to 1.5 kW/m³.

The first depolymerization reactor 211 is a continuously stirred tank reactor and includes any equipment associated with the reactor, such as heat exchangers, control valves, temperature and pressure instrumentation, pumps, compressors, and the like.

The first liquid effluent is removed from the first depolymerization reactor 211 via stream 215 to a first bottoms pump 217. At least a portion of the first liquid effluent is routed to a char handling unit through conduit 223. The remainder of stream 215 is routed as stream 219 to heater 221 and subsequently back to the first depolymerization reactor 211 as a heat input medium. In other embodiments (not shown), the liquid slurry portion recirculated to the reactor is withdrawn from a point of the reactor different from the point of the withdrawal of the liquid slurry portion sent to the char handling. In some embodiments, the liquid slurry is first fed to a dedicated vessel (not shown) equipped with a lower and upper exit point. The liquid portion directed to char handling 233 is withdrawn in a concentrated form the lower exit point while the liquid portion to be recycled 219 to the reactor 211 is withdrawn from the upper exit point.

In some embodiments, heat exchanger 221 heats stream 219 by means of the thermal transfer induced by a flow of molten salt, heated to a temperature in the range of from 300° C. to 570° C. The feeding circuit (not shown) of the molten salt is constructed in such a way to prevent molten salt leakage. The molten salt is molten solar salt preferably constituted by a mixture of sodium nitrate and potassium nitrate, even more preferably in a weight ratio ranging from 2:3 to 3:2. The solar salt receives in turns heat from a dedicated furnace that may be either electric or be fed with fuel. In the latter case, part of the recovered oil from stream 285 from condensation vessel 271 may be used to feed the furnace. In the alternative, or in combination, the heat can be generated by combustion of gaseous or liquid hydrocarbons.

In some embodiments, instead of by external heat exchanger 221, the heat associated to molten salt is transferred to the depolymerization reactor by circulating the molten salt through a jacket which envelops the whole reactor and/or by feeding it to an external heat exchanger described below.

The depolymerization process taking place within the reactor produces molecules having reduced chain length and low boiling point. This continuously running chain breakage mechanism, particularly close to the reactor walls, produces molecules increasingly smaller part of which, at the operating temperature and pressure, are gaseous.

The first gaseous effluent from the first depolymerization reactor 211 is routed via stream 213 to a first condensation vessel 231 from which a second gaseous stream 233 and a second liquid stream 235 are generated and withdrawn. In some embodiments of the process for depolymerizing polymers, the first condensing conditions comprise a temperature in the range of from 20° C. to 250° C., from 30° C. to 200° C., or from 40° C. to 150° C., and a pressure in the range of from 100 kPa to 200 kPa, from 110 kPa to 190 kPa, or from 120 kPa to 180 kPa, or a combination thereof.

In some embodiments, the second gaseous stream 233 is sent to a second condensation vessel 271 working at temperature lower than the first condensation vessel 231. At least a portion of the second liquid stream 235 is sent to a second bottoms pump 237 and is fed as stream 245 to a second depolymerization reactor 251 along with a zeolite catalyst component 207 and optionally a second co-catalyst composition 209.

The zeolite catalyst component is one or more members selected from the group consisting of ZSM-5 zeolites, Beta zeolites, Y zeolites, and ultra-stable Y zeolites, or is one or more ultra-stable Y zeolites.

The amount of zeolite catalyst component 207 used in the present methods can be limited by the requirements of the depolymerization unit. The zeolite catalyst component 207 is a solid that contributes to the dead volume in the unit during the depolymerization reaction. The lower the dead volume, the more polymer that can be depolymerized. In some depolymerization units, the amount of zeolite catalyst component 207 is in the range of from 0.1 wt % to 20 wt %, from 0.2 wt % to 15 wt %, from 0.5 wt % to 10 wt %, or from 1.0 wt % to 8 wt %, wherein weight percentages are based on the total weight of stream 245 the zeolite catalyst component 207, and second co-catalyst component 209 fed to the first depolymerization reactor 251.

The second co-catalyst composition 209 comprises a solid base, an activated clay, or a combination thereof.

In some embodiments, the second co-catalyst composition 209 comprises an activated clay component which is one or more members selected from the group consisting of montmorillonite, sauconite, nontronite, hectorite, beidellite, saponite, bentonite, or a combination thereof, or is bentonite comprising Na-montmorillonite, Ca-montmorillonite, or a combination thereof.

In some embodiments, the second co-catalyst composition 209 comprises a solid base component is one or more members selected from the group consisting of: a) a layered double hydroxide composition; and b) an activated carbon composition. In some embodiments, the second co-catalyst composition 209 comprises a solid base component is one or more members selected from the group consisting of: a) a layered double hydroxide composition; b) an MR composition, wherein M is an alkali metal, an alkaline earth metal, a transition metal, a post-transition metal, metalloid, or a combination thereof, and R is an oxide, a hydroxide, a carbonate, a sulfate, a sulfide, nitrate, nitride, phosphate, phosphite, a halide, or a combination thereof; and c) an activated carbon composition. In some embodiments, the layered double hydroxide is a hydrotalcite, a quintinite, a fougerite, a woodwardite, a cualstibite, a glaucocerinite, a wermlandite, a hydrocalumite, or a combination thereof, or is hydrotalcite. In some embodiments, the MR composition is Ca(OH)₂, Mg(OH)₂, Ba(OH)₂, Sr(OH)₂, CaO, Al₂O₃, or a combination thereof, or is Ca(OH)₂, or a combination thereof.

The amount of second co-catalyst 209 used in the present methods can be limited by the requirements of the depolymerization unit. The second co-catalyst 209 is a solid that contributes to the dead volume in the unit during the depolymerization reaction. The lower the dead volume, the more polymer that can be depolymerized. In some depolymerization units, the amount of second co-catalyst is in the range of from 0.1 wt % to 20 wt %, from 0.2 wt % to 15 wt %, from 0.5 wt % to 10 wt %, or from 1.0 wt % to 8 wt %, wherein weight percentages are based on the total weight of stream 245 the zeolite catalyst component 207, and second co-catalyst component 209 fed to the first depolymerization reactor 251.

In some embodiments, the first co-catalyst component 203 is a solid base, and the second co-catalyst component 209 is an activated clay. In some embodiments, the first co-catalyst component 203 is an activated clay, and the second co-catalyst component 209 is a solid base.

In some embodiments, the second depolymerization reactor 251 is a continuously stirred tank reactor maintained at a temperature ranging from 250° C. to 600° C., from 250° C. to 450° C., from 275° C. to 425° C., or from 300° C. to 400° C., and operated under a pressure ranging from 100 kPa to 1,000 kPa, from 100 kPa to 700 kPa, from 150 kPa to 600 kPa, or from 200 kPa to 500 kPa, in which depolymerization takes place thereby forming a gaseous effluent and a liquid effluent. The remainder, if any, of stream 235 is sent to a first reflux pump 239 and then through cooler 241 and routed back to the first condensation vessel 231 as a gas scrubbing medium.

In some embodiments, the first depolymerization reactor 251 has a cylindrical section with a rounded bottom with a mixer 267 installed in the vertical axis of the reactor 251, completed with a gear motor which allows the blades of the mixer rotating in order to maintain the system in stirred state. The design of the mixer and the power of the motor can vary in respect of the reactor content, volume and shape, however, as a non-limiting example, mixer 227 operates with a power input in the range of from 0.2 kW/m 3 to 4 kW/m³, from 0.25 kW/m 3 to 2 kW/m³, or from 0.3 kW/m 3 to 1.5 kW/m³.

The first depolymerization reactor 251 is a continuously stirred tank reactor and includes any equipment associated with the reactor, such as heat exchangers, control valves, temperature and pressure instrumentation, pumps, compressors, and the like.

A third gaseous effluent stream 253 is withdrawn from the second depolymerization reactor 251 and fed it to the second condensation unit 271 from which a fourth gaseous stream 273 and a fourth liquid stream 275 are generated and withdrawn. In some embodiments, the second condensing conditions independently comprise a temperature in the range of from 20° C. to 100° C., from 30° C. to 90° C., or from 40° C. to 80° C., a pressure in the range of from 30 kPa to 200 kPa, from 50 kPa to 170 kPa, or from 70 kPa to 130 kPa, or a combination thereof.

In some embodiments, at least a portion of the third liquid effluent 255 is recycled to the first depolymerization reactor 211 as stream 265. The portion of the third liquid effluent 255 in excess of stream 265 is routed to a third bottoms pump 257, wherein the discharge of the third bottoms pump 257 is routed to heater 261 and subsequently back to the first depolymerization reactor 211 as a heat input medium via stream 259, to a char handling unit via stream 263, or a combination thereof.

In some embodiments, heat exchanger 261 heats stream 259 by means of the thermal transfer induced by a flow of molten salt, heated to a temperature in the range of from 300° C. to 570° C. The feeding circuit (not shown) of the molten salt is constructed in such a way to prevent molten salt leakage. The molten salt is molten solar salt preferably constituted by a mixture of sodium nitrate and potassium nitrate, even more preferably in a weight ratio ranging from 2:3 to 3:2. The solar salt receives in turns heat from a dedicated furnace that may be either electric or be fed with fuel. In the latter case, part of the recovered oil from stream 285 from condensation vessel 271 may be used to feed the furnace. In the alternative, or in combination, the heat can be generated by combustion of gaseous or liquid hydrocarbons.

At least a portion of the fourth liquid stream 275 is sent to a fourth bottoms pump 283 for discharge of pyrolytic product from the depolymerization processing unit 200. In some embodiments the remainder, if any, of stream 275 is sent to a first reflux pump 277 and then through cooler 281 and routed back to the second condensation vessel 271 as a gas scrubbing medium.

In some embodiments, the amount of first co-catalyst, second co-catalyst, and zeolite catalyst component are each independently in the range of from 0.1 wt % to 20 wt %, from 0.2 wt % to 15 wt %, from 0.5 wt % to 10 wt %, or from 1.0 wt % to 8 wt %, wherein weight percentages are based on the total weight of waste plastic stream 201, the first co-catalyst component 203, second co-catalyst component 209, and the zeolite catalyst component 207, used in process 200. In some embodiments, the composite catalyst system comprises a first co-catalyst 203 and a zeolite catalyst component 207. In some embodiments, the composite catalyst system comprises a second co-catalyst 209 and a zeolite catalyst component 207.

Depending on the type of depolymerization unit, optional additives such as sand may be added to the polyolefin-based feed stream and composite catalyst mixture. Some of these optional additives may contribute to the dead volume of the depolymerization unit, further limiting the amount of the composite catalyst. As an example, a screw kiln depolymerization reactor uses sand as a heat conductor, which limits the amount of dead volume available for the composite catalyst.

The presently disclosed composite catalysts and methods of using them to depolymerize polyolefin-based feed streams are exemplified with respect to the examples below. These examples are included to demonstrate embodiments of the appended Claims. However, these are exemplary only, and the invention can be broadly applied to any combination of polyolefin-based feed, with and without non-polyolefin components, and composite catalyst. Those of skill in the art should appreciate that many changes can be made in the specific embodiments which are disclosed and still obtain a like or similar result without departing from the spirit and scope of the disclosure herein. In no way should the following examples be read to limit, or to define, the scope of the appended claims.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Raw Materials

A polyolefin-based feeds (“PBF1” and “PBF2”) comprising impurities were used in Examples 1-76 to evaluate the depolymerization performance of certain zeolite catalysts, activated clays, solid bases, or combinations thereof. The polyolefin-based feed “PBF1” comprised a mixture of approximately equal amount of postconsumer recyclate comprising 1:1 ratio of polyethylene and polypropylene, along with non-polyolefinic components of 5 wt % ash, 0.9 wt % water, 0.8 wt % polystyrene, 0.3 wt % polyethylene terephthalate, and 0.3 wt % chlorine, wherein all weight percentages are based on the total weight of PBF1. The polyolefin-based feed “PBF2” comprised a mixture of approximately equal amount of postconsumer recyclate comprising 1:1 ratio of polyethylene and polypropylene, along with non-polyolefinic components of 7 wt % ash, 1 wt % water, 0.5 wt % polystyrene, 4 wt % polyethylene terephthalate, and 0.3 wt % chlorine, wherein all weight percentages are based on the total weight of PBF2.

Zeolite catalysts used herein are shown in Table 1, below. SiO₂/Al₂O₃ mole ratio (“SAR”) are shown for each catalyst.

TABLE 1

| Zeolite | | | |
|---|---|---|---|
| Type | Grade | SAR | Available from |
| Beta | CP811E-75 | 75 | Zeolyst, Valley Forge, PA |
| Beta | CP811TL | 40 | Zeolyst, Valley Forge, PA |
| Beta | CP7119 | 26 | Zeolyst, Valley Forge, PA |
| Beta | CP811C-300 | 300 | Zeolyst, Valley Forge, PA |
| Beta | CP811T-100 | 100 | Zeolyst, Valley Forge, PA |
| H-ZSM-5 | CBV3014 | 30 | Zeolyst, Valley Forge, PA |
| H-ZSM-5 | CBV3024E | 30 | Zeolyst, Valley Forge, PA |
| H-ZSM-5 | CBV2314 | 23 | Zeolyst, Valley Forge, PA |
| H-ZSM-5 | CBV8014 | 80 | Zeolyst, Valley Forge, PA |
| H-ZSM-5 | CBV5524G | 50 | Zeolyst, Valley Forge, PA |
| H-ZSM-5 | ZD13008 | 80 | Zeolyst, Valley Forge, PA |
| H-ZSM-5 | CBV28014 | 280 | Zeolyst, Valley Forge, PA |
| H-ZSM-5 | CBV15014G | 150 | Zeolyst, Valley Forge, PA |
| H-USY | CFG-1 | 13.4 | Zeolyst, Valley Forge, PA |
| H-Y | CBV-300 | 5.1 | Zeolyst, Valley Forge, PA |
| H-Y | CBV-400 | 5.1 | Zeolyst, Valley Forge, PA |
| H-Y | CBV-500 | 5.2 | Zeolyst, Valley Forge, PA |
| H-Y | CBV-712 | 12 | Zeolyst, Valley Forge, PA |

Co-catalysts used herein are shown in Table 2, below.

TABLE 2

| Composition | Grade | Co-catalyst type | Available from |
|---|---|---|---|
| Bentonite, acid treated | F20X | Activated clay | EP Engineered Clays Corporation, Jackson, MS |
| Halloysite | n/a | Activated clay | Sigma Aldrich, St. Louis, MO |
| Bentonite, acid treated | CC105SF | Activated clay | EP Engineered Clays Corporation, Jackson, MS |
| Bentonite, acid treated | CC170 | Activated clay | EP Engineered Clays Corporation, Jackson, MS |
| Bentonite, acid treated | F20XLM | Activated clay | EP Engineered Clays Corporation, Jackson, MS |
| Bentonite, acid treated | F21 | Activated clay | EP Engineered Clays Corporation, Jackson, MS |
| Bentonite, acid treated | F30X | Activated clay | EP Engineered Clays Corporation, Jackson, MS |
| Bentonite, acid treated | EX1314III | Activated clay | Clariant Corporation, Pasadena, TX |
| Bentonite, acid treated | EX831 | Activated clay | Clariant Corporation, Pasadena, TX |
| acid-leached phyllosilicate | Fulcat 22F | Activated clay | BYK USA Inc., Chester, NY |
| catalyst¹ | | | |
| acid-leached phyllosilicate | Fulcat 435 | Activated clay | BYK USA Inc., Chester, NY |
| catalyst¹ | | | |
| bleaching earth | Tonsil Supreme | Activated clay | Clariant Corporation, Pasadena, TX |
| | 115FF | | |
| Ca(OH)₂ | base | Solid base | Sigma Aldrich, St. Louis, MO |
| Act Carbon, , | NORIT ™ GAC | Activated clay | Cabot Corporation, Franklin, LA |
| | 400, adsorbent | | |
| hydrotalcite, synthesized | base | Solid base | Sigma Aldrich, St. Louis, MO |
| Zr(HPO₄)₂ | adsorbent | Activated clay | Sigma Aldrich, St. Louis, MO |
| Bentonite, acid treated | M300F | Activated clay | Sepigel, Guadalajara, Spain |
| Bentonite, acid treated | F21X | Activated clay | EP Engineered Clays Corporation, Jackson, MS |
| acid-leached phyllosilicate | Fulcat 22B | Activated clay | BYK USA Inc., Chester, NY |
| catalyst¹ | | | |
| Bentonite, acid treated | M200UF | Activated clay | Sepigel, Guadalajara, Spain |
| Bentonite, acid treated | F20X | Activated clay | EP Engineered Clays Corporation, Jackson, MS |
| Bentonite, acid treated | Zakuro Regular | Activated clay | Musim Mas, Singapore |
| | Plus | | |
| Bentonite, acid treated | Zakuro Super | Activated clay | Musim Mas, Singapore |

¹with active Lewis and Brønsted acid sites

### Experimental Process

Unless other noted, the depolymerization unit was a Thermogravimetric Gravimetric Analysis (TGA) instrument. For the TGA thermolysis reactions, the uniform samples were heated under nitrogen at 10K/yin to a depolymerization temperature of 400° C. in a Mettler Toledo TGA/DSC 3+(Mettler Toledo, Columbus, Ohio) and held for 1 hour. The &polymerization half time at a specific temperature, defined as the time needed to achieve a 50% loss of mass, was recorded directly if the value was less than 60 min., or determined under the assumption of first order decomposition kinetics as t_{1/2}=0.693/k, where k is the first order rate constant determined graphically using a Ln(C₀/C) vs time plot.

The depolymerization half time is related to the residence time needed in a large scale depolymerization unit. The shorter the half time, the shorter the residence time for a batch of a polymer feed in a depolymerization unit, and the faster the depolymerization rate k.

The samples were prepared by melt-compounding with the composite catalyst in a HAAK MiniCTW compounder at 200° C. and 200 RPM for 5 minutes. Each composition and its depolymerization half time are shown in Tables 3-9.

### EXAMPLES 1-4

Table 3 summarizes the results of Examples 1-4. Examples 1˜4 are each performed with a 5 g sample of PBF1. Example 1 provides benchmark data for depolymerization of PBF1 without a zeolite catalyst, a solid base co-catalyst, or an activated clay co-catalyst. Example 2 shows that, relative to Example 1, t_{1/2} increases by 79% when only activated clay is added. Example 3 shows that, relative to Example 1, t_{1/2} increases by 34% when only solid base is added to the reactor. Example 4 shows that, relative to Example 1, t_{1/2} increases by 34% when solid base and activated clay is added to the reactor.

Without wishing to be bound by any particular theory, it is believed that Example 4 shows a 25% reduction in t_{1/2} as compared to Example 2 since the addition of the solid base co-catalyst in the reactor provides a poison mitigation effect such that the catalytic reaction with the weak acid activated clay co-catalyst in the reactor.

TABLE 3

| | Catalyst | | Co-catalyst(s) | | | | | |
|---|---|---|---|---|---|---|---|---|
| | Zeolite | Mass | | Mass | | Mass | k; | t_{1/2}, |
| Ex. | Catalyst | (g) | Clay | (g) | Base | (g) | 400° C. | (min.) |
| 1 | none | none | none | none | none | none | 0.0075 | 92.4 |
| 2 | none | none | Bentonite F20X | 0.2 | none | none | 0.0042 | 165.0 |
| 3 | none | none | none | none | Ca(OH)₂ | 0.2 | 0.0056 | 123.8 |
| 4 | none | none | Bentonite F20X | 0.2 | Ca(OH)₂ | 0.2 | 0.0056 | 123.8 |

### EXAMPLES 5-12

Table 4 summarizes the results of Examples 5-12. Examples 5-12 are each performed with a 5 g sample of PBF1. Example 5 provides benchmark data for depolymerization of PBF1 in the reactor with only a beta zeolite catalyst. Example 6 shows that, relative to Example 5, t_{1/2} decreases by 23% when only solid base is added to the reactor. Example 7 shows that, relative to Example 5, t_{1/2} decreases by 25% when only activated clay is added to the reactor. Examples 8-10 show that addition of the solid base with an activated clay and zeolite in the reactor reduces the t_{1/2} time versus addition of either the solid base (Example 6) or the activated clay (Example 7) in conjunction with the zeolite catalyst.

Without wishing to be bound by any particular theory, it is believed that Examples 11 and 12 do not perform as well as Examples 8-10 due to the beta zeolites of Examples 11 and 12 having a significantly higher silica-to-alumina mole ratio (“SAR”).

TABLE 4

| | Catalyst | | Co-catalyst(s) | | | | | |
|---|---|---|---|---|---|---|---|---|
| | Zeolite | Mass | | Mass | | Mass | k; | t_{1/2}, |
| Ex. | Catalyst | (g) | Clay | (g) | Base | (g) | 400° C. | (min.) |
| 5 | beta | 0.1 | none | none | none | none | 0.0057 | 121.6 |
| | (CP811E75) | | | | | | | |
| 6 | beta | 0.1 | none | none | Ca(OH)₂ | 0.2 | 0.0074 | 93.6 |
| | (CP811E75) | | | | | | | |
| 7 | beta | 0.1 | Bentonite | 0.2 | none | none | 0.0076 | 91.2 |
| | (CP811E75) | | F20X | | | | | |
| 8 | beta | 0.1 | Bentonite | 0.2 | Ca(OH)₂ | 0.1 | 0.0147 | 47.1 |
| | (CP811E75) | | F20X | | | | | |
| 9 | beta | 0.1 | Bentonite | 0.2 | Ca(OH)₂ | 0.1 | 0.0097 | 71.4 |
| | (CP811TL) | | F20X | | | | | |
| 10 | beta | 0.1 | Bentonite | 0.2 | Ca(OH)₂ | 0.1 | 0.0091 | 76.2 |
| | (CP7119) | | F20X | | | | | |
| 11 | beta | 0.1 | Bentonite | 0.2 | Ca(OH)₂ | 0.1 | 0.0057 | 121.6 |
| | (CP811C300) | | F20X | | | | | |
| 12 | beta | 0.1 | Bentonite | 0.2 | Ca(OH)₂ | 0.1 | 0.0056 | 123.8 |
| | (CP811T100) | | F20X | | | | | |

### EXAMPLES 13-23

Table 5 summarizes the results of Examples 13-23. Examples 13-23 are each performed with a 5 g sample of PBF1. Example 13 provides benchmark data for depolymerization of PBF1 in the reactors with only a ZSM-5 zeolite catalyst. Example 15 shows that, relative to Example 13, t_{1/2} decreases by 49% when only solid base is added to the first micro-reactor. Example 14 shows that, relative to Example 13, t_{1/2} decreases by 31% when only activated clay is added to the reactor. Example 16 show that addition of the solid base in conjunction with an activated clay and the same ZSM-5 zeolite in the reactor reduces the t_{1/2} time versus addition of either the solid base (Example 15) or the activated clay (Example 14) in conjunction with the zeolite catalyst.

Without wishing to be bound by any particular theory, it is believed that Examples 17-23 show a trend of increasing t_{1/2} relative to Example 16 correlates to the increasing SAR of these ZSM-5 zeolites.

TABLE 5

| | Catalyst | | Co-catalyst(s) | | | | | |
|---|---|---|---|---|---|---|---|---|
| | Zeolite | Mass | | Mass | | Mass | k; | t_{1/2}, |
| Ex. | Catalyst | (g) | Clay | (g) | Base | (g) | 400° C. | (min.) |
| 13 | H-ZSM-5 (CBV3014) | 0.1 | none | none | none | none | 0.0082 | 84.5 |
| 14 | H-ZSM-5 (CBV3014) | 0.1 | Bentonite F20X | 0.2 | none | none | 0.0119 | 58.2 |
| 15 | H-ZSM-5 (CBV3014) | 0.1 | none | none | Ca(OH)₂ | 0.2 | 0.016 | 43.3 |
| 16 | H-ZSM-5 (CBV3014) | 0.1 | Bentonite F20X | 0.2 | Ca(OH)₂ | 0.1 | 0.0338 | 20.5 |
| | SAR 20 | | | | | | | |
| 17 | H-ZSM-5 (CBV3024E) | 0.1 | Bentonite F20X | 0.2 | Ca(OH)₂ | 0.1 | 0.0153 | 45.3 |
| 18 | H-ZSM-5 (CBV2314) | 0.1 | Bentonite F20X | 0.2 | Ca(OH)₂ | 0.1 | 0.0141 | 49.1 |
| 19 | H-ZSM-5 (CBV8014) | 0.1 | Bentonite F20X | 0.2 | Ca(OH)₂ | 0.1 | 0.0094 | 73.7 |
| 20 | H-ZSM-5 | 0.1 | Bentonite F20X | 0.2 | Ca(OH)₂ | 0.1 | 0.0085 | 81.5 |
| | (CBV5524G) | | | | | | | |
| 21 | H-ZSM-5 (ZD13008) | 0.1 | Bentonite F20X | 0.2 | Ca(OH)₂ | 0.1 | 0.0061 | 113.6 |
| 22 | H-ZSM-5 (CBV28014) | 0.1 | Bentonite F20X | 0.2 | Ca(OH)₂ | 0.1 | 0.0059 | 117.5 |
| 23 | H-ZSM-5 | 0.1 | Bentonite F20X | 0.2 | Ca(OH)₂ | 0.1 | 0.0058 | 119.5 |
| | (CBV15014G) | | | | | | | |

### EXAMPLES 24-44 and 86-95

Table 6 summarizes the results of Examples 24-56 and 86-95. Examples 24-56 and 86-95 are each performed with a 5 g sample of PBF1 and demonstrate use of 0.1-0.2 g of an ultra-stable Y (HUSY) zeolite catalyst in the reactor. Examples 24 and 27 provide benchmark data for depolymerization of PBF1 in the first and second micro-reactors with only an HUSY zeolite catalyst in the second micro-reactor. Examples 26, 29-56, and 86-95 show that, relative to Examples 24 and 27, t_{1/2} decreases with addition of activated clay co-catalyst to the reactor.

Without wishing to be bound by any particular theory, it is believed that the lesser combined amount of catalyst and co-catalyst in Examples 25 and 28 was not enough to overcome the negative effects of impurities in the polyolefin-based feed. It is further believed that in a commercial continuous operation of two reactors in series, all results would be improved since pyrolysis in the first reactor and removal of a char waste stream would result in removal of some portion of catalyst poisons being removed from the product from the first reactor and consequently the feed to the second reactor.

TABLE 6

| | Catalyst | | Co-catalyst(s) | | | | | |
|---|---|---|---|---|---|---|---|---|
| | Zeolite | Mass | | Mass | | Mass | k; | t_{1/2}, |
| Ex. | Catalyst | (g) | Clay | (g) | Base | (g) | 400° C. | min |
| 24 | HUSY (CFG-1) | 0.1 | none | none | none | none | 0.0074 | 93.6 |
| 25 | HUSY (CFG-1) | 0.1 | Bentonite F20X | 0.2 | none | none | 0.0048 | 144.4 |
| 26 | HUSY (CFG-1) | 0.1 | Bentonite F20X | 0.4 | none | none | 0.0153 | 45.3 |
| 27 | HUSY (CFG-1) | 0.2 | none | none | none | none | 0.0075 | 92.4 |
| 28 | HUSY (CFG-1) | 0.2 | Bentonite F20X | 0.1 | none | none | 0.0065 | 106.6 |
| 29 | HUSY (CFG-1) | 0.2 | Halloysite | 0.1 | none | none | 0.0099 | 70.0 |
| 30 | HUSY (CFG-1) | 0.2 | Zr(HPO₄)₂ | 0.1 | none | none | 0.0259 | 26.8 |
| 31 | HUSY (CFG-1) | 0.2 | Bentonite F20X | 0.2 | none | none | 0.0186 | 37.3 |
| 32 | HUSY (CFG-1) | 0.2 | Bentonite CC105SF | 0.2 | none | none | 0.0189 | 36.7 |
| 33 | HUSY (CFG-1) | 0.2 | Bentonite CC170 | 0.2 | none | none | 0.011 | 63.0 |
| 34 | HUSY (CFG-1) | 0.2 | Bentonite F20XLM | 0.2 | none | none | 0.0297 | 23.3 |
| 35 | HUSY (CFG-1) | 0.2 | Bentonite F21 | 0.2 | none | none | 0.0268 | 25.9 |
| 36 | HUSY (CFG-1) | 0.2 | Bentonite F30X | 0.2 | none | none | 0.0212 | 32.7 |
| 37 | HUSY (CFG-1) | 0.2 | Fulcat22F | 0.2 | none | none | 0.0281 | 24.7 |
| 38 | HUSY (CFG-1) | 0.2 | Fulcat435 | 0.2 | none | none | 0.0276 | 25.1 |
| 39 | HUSY (CFG-1) | 0.2 | Halloysite | 0.2 | none | none | 0.0123 | 56.3 |
| 40 | HUSY (CFG-1) | 0.2 | Zr(HPO₄)₂ | 0.2 | none | none | 0.0368 | 18.8 |
| 41 | HUSY (CFG-1) | 0.2 | Tonsil Supr. 115FF | 0.2 | none | none | 0.0221 | 31.4 |
| 42 | HUSY (CFG-1) | 0.2 | Bentonite F20X | 0.3 | none | none | 0.0193 | 35.9 |
| 43 | HUSY (CFG-1) | 0.2 | Halloysite | 0.4 | none | none | 0.0288 | 24.1 |
| 44 | HUSY (CFG-1) | 0.2 | Bentonite F20X | 0.4 | none | none | 0.0257 | 27.0 |
| 86 | HUSY (CFG-1) | 0.2 | EX381 | 0.2 | none | none | 0.0151 | 46.0 |
| 87 | HUSY (CFG-1) | 0.2 | EX1314III | 0.2 | none | none | 0.0196 | 35.0 |
| 88 | HUSY (CFG-1) | 0.2 | Zakuro Regular Plus | 0.1 | none | none | 0.014 | 50.0 |
| 89 | HUSY (CFG-1) | 0.2 | Zakuro Regular Plus | 0.2 | none | none | 0.0233 | 30.0 |
| 90 | HUSY (CFG-1) | 0.2 | Zakuro Regular Plus | 0.4 | none | none | 0.0294 | 24.0 |
| 91 | HUSY (CFG-1) | 0.2 | Zakuro Super | 0.1 | none | none | 0.0128 | 54.0 |
| 92 | HUSY (CFG-1) | 0.2 | Zakuro Super | 0.2 | none | none | 0.0228 | 30.0 |
| 93 | HUSY (CFG-1) | 0.2 | Zakuro Super | 0.4 | none | none | 0.0293 | 24.0 |
| 94 | HUSY (CFG-1) | 0.2 | Zakuro Regular Plus | 0.2 | none | none | 0.0193 | 36.0 |
| 95 | HUSY (CFG-1) | 0.2 | Zakuro Super | 0.2 | none | none | 0.0328 | 21.0 |

### Examples 45-51

Table 7 summarizes the results of Examples 45-51. Examples 24 and 27 are copied from Table 6 to provide benchmark data for depolymerization of PBF1 in the first and second micro-reactors with only an HUSY zeolite catalyst in the reactor. Examples 45-51 are each performed with a 5 g sample of PBF1 and demonstrate use of 0.2 g of an HUSY zeolite catalyst in the reactor in conjunction with the use of different amounts of different solid bases in the first micro-reactor. Examples 45-47 and 49-51 all show a substantial reduction in t_{1/2} times relative to Examples 24 and 27 due to the absence of clay component.

Without wishing to be bound by any particular theory, it is believed that the increase in the t_{1/2} time in Example 48 relative to Examples 24 and 27 is due to the need for a clay component along with the particular solid base in order to achieve the desired effect. It is further believed that in a commercial continuous operation of two reactors in series, all results would be improved since pyrolysis in the first reactor and removal of a char waste stream would result in removal of some portion of catalyst poisons being removed from the product from the first reactor and consequently the feed to the second reactor.

TABLE 7

| | Catalyst | | Co-catalyst(s) | | | | | |
|---|---|---|---|---|---|---|---|---|
| | Zeolite | Mass | | Mass | | Mass | k; | t_{1/2}, |
| Ex. | Catalyst | (g) | Clay | (g) | Base | (g) | 400° C. | min |
| 24 | HUSY (CFG-1) | 0.1 | none | none | none | none | 0.0074 | 93.6 |
| 27 | HUSY (CFG-1) | 0.2 | none | none | none | none | 0.0075 | 92.4 |
| 45 | HUSY (CFG-1) | 0.2 | none | none | Ca(OH)₂ | 0.05 | 0.0083 | 83.5 |
| 46 | HUSY (CFG-1) | 0.2 | none | none | Ca(OH)₂ | 0.1 | 0.0112 | 61.9 |
| 47 | HUSY (CFG-1) | 0.2 | none | none | Act Carbon | 0.1 | 0.007 | 99.0 |
| 48 | HUSY (CFG-1) | 0.2 | none | none | hydrotalcite | 0.1 | 0.0064 | 108.3 |
| 49 | HUSY (CFG-1) | 0.2 | none | none | Ca(OH)₂ | 0.2 | 0.0162 | 42.8 |
| 50 | HUSY (CFG-1) | 0.2 | none | none | Ca(OH)₂ | 0.3 | 0.0144 | 48.1 |
| 51 | HUSY (CFG-1) | 0.2 | none | none | Ca(OH)₂ | 0.4 | 0.0108 | 64.2 |

### EXAMPLES 52-69 and 96

Table 8 summarizes the results of Examples 52-69 and 96. Examples 52-69 and 96 are each performed with a 5 g sample of PBF1 and demonstrate use of 0.1-0.3 g of an HUSY zeolite catalyst in the reactor in conjunction with the use of different amounts of different solid bases in the reactor. When compared to results in Table 6 (HUSY zeolite and activated clay in reactor) and Table 7 (HUSY zeolite in reactor in conjunction with solid base), Examples 52-69 and 96 in Table 8 (HUSY zeolite and activated clay in second micro-reactor in conjunction with solid base in reactor) show a trend of reducing t_{1/2} times more when both co-catalysts are used than when either co-catalyst is used individually.

Examples 55, 57, and 61 show the highest t_{1/2} times for HUSY zeolites used conjunction with both co-catalysts. However, Examples 55 (activated carbon as sold base) and 57 (hydrotalcite as solid base) demonstrated t_{1/2} times of 45 minutes and 44.7 minutes, respectively. Examples 47 (activated carbon as sold base) and 48 (hydrotalcite as solid base) in Table 7 demonstrated t_{1/2} times of 99.0 minutes and 108.3 minutes, respectively. Therefore, when comparing similar types of solid bases, addition of the activated clay co-catalyst in the second micro-reactor reduced t_{1/2} times by over 50%. Although Example 61 (halloysite as activated clay) shows a higher t_{1/2} time than Example 29 in Table 6, it is believed that in a commercial continuous operation of two reactors in series, the use of a solid base in the first reactor in combination with halloysite in the second reactor would result in better conversion than produced in the micro-reactors, since removal of a char waste stream from the first reactor would result in removal of some portion of catalyst poisons being removed from the product from the first reactor and consequently the feed to the second reactor.

TABLE 8

| | Catalyst | | Co-catalyst(s) | | | | | |
|---|---|---|---|---|---|---|---|---|
| | Zeolite | Mass | | Mass | | Mass | k; | t_{1/2}, |
| Ex. | Catalyst | (g) | Clay | (g) | Base | (g) | 400° C. | min |
| 52 | HUSY (CFG-1) | 0.1 | Bentonite F20X | 0.2 | Ca(OH)₂ | 0.1 | 0.0144 | 48.1 |
| 53 | HUSY (CFG-1) | 0.1 | Bentonite F20X | 0.2 | Ca(OH)₂ | 0.2 | 0.013 | 53.3 |
| 54 | HUSY (CFG-1) | 0.1 | Bentonite F20X | 0.4 | Ca(OH)₂ | 0.1 | 0.0178 | 38.9 |
| 55 | HUSY (CFG-1) | 0.2 | Bentonite F20X | 0.1 | Act Carbon | 0.1 | 0.0154 | 45 |
| 56 | HUSY (CFG-1) | 0.2 | Bentonite F20X | 0.1 | Ca(OH)₂ | 0.1 | 0.0172 | 40.3 |
| 57 | HUSY (CFG-1) | 0.2 | Bentonite F20X | 0.1 | hydrotalcite | 0.1 | 0.0155 | 44.7 |
| 58 | HUSY (CFG-1) | 0.2 | Bentonite F20X | 0.1 | Ca(OH)₂ | 0.3 | 0.0257 | 27.0 |
| 59 | HUSY (CFG-1) | 0.2 | Bentonite F20X | 0.2 | Ca(OH)₂ | 0.1 | 0.0331 | 20.9 |
| 60 | HUSY (CFG-1) | 0.2 | Bentonite F20X | 0.2 | Ca(OH)₂ | 0.2 | 0.0279 | 24.8 |
| 61 | HUSY (CFG-1) | 0.2 | Halloysite | 0.2 | Ca(OH)₂ | 0.2 | 0.0099 | 70.0 |
| 62 | HUSY (CFG-1) | 0.2 | Bentonite F20X | 0.2 | Ca(OH)₂ | 0.3 | 0.0246 | 28.2 |
| 63 | HUSY (CFG-1) | 0.2 | Bentonite F20X | 0.3 | Ca(OH)₂ | 0.1 | 0.0295 | 23.5 |
| 64 | HUSY (CFG-1) | 0.2 | Bentonite F20X | 0.3 | Ca(OH)₂ | 0.2 | 0.0262 | 26.5 |
| 65 | HUSY (CFG-1) | 0.2 | Bentonite F20X | 0.3 | Ca(OH)₂ | 0.3 | 0.0302 | 22.9 |
| 66 | HUSY (CFG-1) | 0.2 | Bentonite F20X | 0.4 | Ca(OH)₂ | 0.1 | 0.0392 | 17.7 |
| 67 | HUSY (CFG-1) | 0.2 | Bentonite F20X | 0.4 | Ca(OH)₂ | 0.2 | 0.04 | 17.3 |
| 68 | HUSY (CFG-1) | 0.2 | Bentonite F20X | 0.4 | Ca(OH)₂ | 0.2 | 0.0384 | 18.0 |
| 69 | HUSY (CFG-1) | 0.3 | Bentonite F20X | 0.1 | Ca(OH)₂ | 0.1 | 0.0328 | 21.1 |
| 96 | HUSY (CFG-1) | 0.2 | Bentonite F20X | 0.1 | Ca(OH)₂ | 0.2 | 0.0229 | 30.0 |

### Examples 70-73

Table 9 summarizes the results of Examples 70-73. Examples 70-73 are each performed with a 5 g sample of PBF1 and demonstrate use of 0.2 g of a number of different Y zeolite catalysts with both an activated clay and a solid base in the reactor. Again, use of both co-catalysts these different Y zeolites shows similar performance to other zeolites used in conjunction with both co-catalysts.

TABLE 9

| | Catalyst | | Co-catalyst(s) | | | | | |
|---|---|---|---|---|---|---|---|---|
| | Zeolite | Mass | | Mass | | Mass | k; | t_{1/2}, |
| Ex. | Catalyst | (g) | Clay | (g) | Base | (g) | 400° C. | (min.) |
| 70 | H-Y (CBV300) | 0.1 | Bentonite F20X | 0.2 | Ca(OH)₂ | 0.1 | 0.0129 | 53.7 |
| 71 | H-Y (CBV400) | 0.1 | Bentonite F20X | 0.2 | Ca(OH)₂ | 0.1 | 0.0137 | 50.6 |
| 72 | H-Y (CBV500) | 0.1 | Bentonite F20X | 0.2 | Ca(OH)₂ | 0.1 | 0.014 | 49.5 |
| 73 | H-Y (CBV712) | 0.1 | Bentonite F20X | 0.2 | Ca(OH)₂ | 0.1 | 0.0346 | 20.0 |

### Examples 74-76

Table 10 summarizes the results of Examples 74-76. Examples 74-76 were conducted using the Frontier Lab tandem micro-reactor system (model no.: Rx-3050TR) connected to an Agilent GC/MS (model nos. 8890 & 5977). The feed used in Examples 74-76 was waste feed PBF2. The zeolite used in these examples was HUSY (CFG-1). The activated clay used in these examples was Bentonite F20X. The solid base used in these examples was Ca(OH)₂.

These examples demonstrate the value of staging the co-catalysts with a system using two pyrolysis reactors in series. The first microreactor was operated at a temperature of 500° C. and pressure of 13 psig. The second microreactor was operated at a temperature of 300° C. and pressure of 13 psig.

Example 74 shows the depolymerization conversion performance using only zeolite catalyst in the second reactor. Example 75 shows improved depolymerization conversion performance over that of Example 74, achieved by adding activated clay and solid base co-catalysts in the first reactor in conjunction with zeolite catalyst in the second reactor. Example 76 shows further improved depolymerization conversion performance over that of Example 75, achieved by adding only solid base co-catalyst in the first reactor in conjunction with activated clay co-catalyst and zeolite catalyst in the second reactor.

TABLE 10

| | First Reactor | | | Second Reactor | |
|---|---|---|---|---|---|
| Ex. | Polymer | Base | Clay | Zeolite | Clay |
| 74 | PBF2 | — | — | 2.5 wt % | — |
| 75 | PBF2 | 2.5 wt % | 2.5 wt % | 2.5 wt % | — |
| 76 | PBF2 | 2.5 wt % | — | 2.5 wt % | 2.5 wt % |

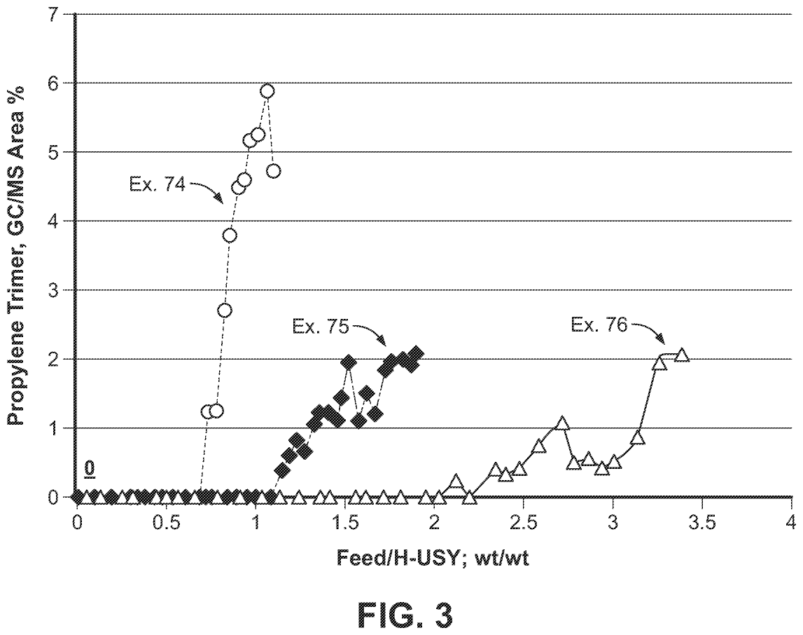

FIG. 3 shows a graphical comparison of Examples 74-76 at conditions described above and shown in Table 10. The catalytic efficiency was measured by the level of propylene trimer in the product stream, wherein lower values of propylene trimer content correlate to higher levels of catalyst activity, and higher values of propylene trimer content correlate to lower levels of catalyst activity. Propylene trimer content measurements, calculated as percentage of the total mass spectrometry (MS) area for the first 30 minutes of the run, in Examples 74-76 are shown by the y-axis values in FIG. 3. Effective catalyst life is shown by the amount of plastic waste processed per unit of zeolite catalyst. Weight of feed per weight of catalyst measurements in Examples 74-76 are shown by the x-axis values in FIG. 3.

Comparison of Example 75 to Example 74 shows that addition of the clay and base co-catalysts both improve catalyst life and catalyst efficiency. Comparison of Example 76 to Example 75 shows that addition of the base co-catalysts before the clay co-catalyst further improves both catalyst life and catalyst efficiency.

### EXAMPLE(S) 77-84

### Raw Materials for Example(s) 77-84

A post-consumer polyolefin-based feed (“PBF3”) comprising impurities was used in Examples 80-87 to evaluate the depolymerization performance of certain activated clays. The polyolefin-based feed “PBF3” comprised a mixture of approximately 97 wt % of a 30/70 weight ratio of polypropylene to polyethylene (PP:PE) with the remainder containing traces of other common polymers (polyethylene terephthalate, polystyrene, polyamide, and polyurethane) and inorganic contaminants.

Experimental Process for Example(s) 77-84

Thirty (30) grams of PBF3 was loaded into a 500 mL round glass reactor having three necks equipped with thermocouple and nitrogen inlet. Two and a half weight percent (2.5 wt %), based on the weight of PBF3, of a solid catalyst, a H-Y zeolite catalyst (CBV400, Zeolyst International), was added to the reactor for each of Examples 77-84. In each of Examples 78-84, two and a half weight percent (2.5 wt %), based on the weight of PBF3, of the co-catalyst indicated in Table 12 was also added to the reactor. Two glass condensers were connected in series to the third neck of the reactor and maintained at 110° C. and −8° C. respectively using an oil bath (Cryostat Julabo). The reactor was placed in an electrical heating system (i.e., a mantle bath). The samples were heated under nitrogen at 10K/min to a depolymerization temperature of 430° C. and held for two hours.

TABLE 12

| | | | | | Efficiency of |
|---|---|---|---|---|---|
| | | | | | Co-Catalyst as % |
| | | | | | Solids Residue |
| | Catalyst | | Co-Catalyst | | Reduction |
| | Zeolite | | Activated | | Normalized |
| Ex. | Catalyst | Wt % | Clay | Wt % | vs. Ex. 77 |
| 77 | H-Y(CBV400) | 2.5 | None | 0 | — |
| 78 | H-Y(CBV400) | 2.5 | Fulcat 435 | 2.5 | 77% |
| 79 | H-Y(CBV400) | 2.5 | Tonsil Supreme | 2.5 | 77% |
| | | | 115FF | | |
| 80 | H-Y(CBV400) | 2.5 | M300UF | 2.5 | 69% |
| 81 | H-Y(CBV400) | 2.5 | F21X | 2.5 | 54% |
| 82 | H-Y(CBV400) | 2.5 | Fulcat 22B | 2.5 | 42% |
| 83 | H-Y(CBV400) | 2.5 | M200UF | 2.5 | 38% |
| 84 | H-Y(CBV400) | 2.5 | F20X | 2.5 | 19% |

Thereafter, the pyrolysis oil, gaseous products, and solid residue remaining in the reactor were collected and measured. Table 12 summarizes the efficiency of the co-catalysts as a percentage of the solids residue reduction normalized to the results of Example 77. As evident from Table 12, the systems including the Fulcat 435, Tonsil Supreme 115FF, and M300UF co-catalysts provided the greatest reduction in residual solids.

### EXAMPLE 85

The following experimental steps were carried out in a depolymerization apparatus of two reactors connected in series, consisting of a mechanically agitated vessel that is jacketed for heating. The first reactor was provided with an inlet for the plastic waste coming from the extruder feed, and an outlet for the generated gases. The gases withdrawn from the reactor are conveyed to a condensation unit from which an incondensable gas and a pyrolytic oil are obtained. Thermocouples are positioned into the reactor to monitor and record the temperatures. The oil collected from the condensation unit is fed to a second depolymerization reactor, provided also with an inlet for catalyst feeding The catalyst was fed into the reactor as solid slurry by mixing with part of the same oil from the condensation section.

The second reactor was also provided with an outlet line in order to recycle part of the reactor content to the first depolymerization reactor.

The polyolefin-based feed “PFB4” comprised a mixture of postconsumer PE and PP at a 2:1 weight ratio along with non-polyolefinic components of 8 wt % ash, 1 wt % polystyrene, and 1 wt % nylon 6 wherein all weight percentages are based on the total weight of PFB4.

The PFB4 feedstock was homogenized and pelletized before the loading in the hopper needed to feed the extruder which worked at a temperature of 290° C. and discharged continuously into the depolymerization reactor at 4 kg/h. The first depolymerization reactor was operated at a pressure of 4 barg and at temperature of about 412° C. while the average residence time was about 192 minutes. The gaseous phase of the reactor was sent to a condensation unit formed by a cooling/scrubber column working at 80° C. and a dephlegmator working at 25° C. Then, the oil stream was fed into the second vessel operated at 335° C. and 5.5 barg. Average residence time in this case was about 138 minutes. In this second reactor, a sample of equal parts H-USY Zeolite type (CFG-1, Zeolyst International) and activated clay (Fulcat 435, BYK USA Inc.) was tested. The catalyst mixture was fed into the pyrolizer in such an amount to get a ratio of 6 wt % with respect to the reactive phase mass.

TABLE 11

| | Rx1 | Rx1 | Rx1 | Rx2 | Rx2 | Rx2 | Solid | PyOil | Gas |
|---|---|---|---|---|---|---|---|---|---|
| Feedstock | T(° C.) | p(barg) | tau(h) | T(° C.) | P(barg) | tau(h) | (wt %) | (wt %) | (wt %) |
| PFB4 | 412 | 4 | 3.2 | 335 | 5.5 | 2.3 | 11 | 54 | 35 |

Rx1 T(° C.) = Reactor 1 − Pyrolysis TemperaturePFG
Rx1 p(barg) = Reactor 1 − Pyrolysis Pressure
Rx1 tau (min) = Reactor 1 − Pyrolysis Residence Time
Rx2 T(° C.) = Reactor 2 − Pyrolysis Temperature
Rx2 p(barg) = Reactor 2 − Pyrolysis Pressure
Rx2 tau (min) = Reactor 2 − Pyrolysis Residence Time
Solid (wt %) = residue in the reactor with respect to the feedstock
Pyoil (wt %) = yield in liquid with respect to feedstock
Gas (wt %) = yield in gas with respect to feedstock

Table 11 summarizes the process conditions and results of Example 85 showing that the product comprises: 54 wt % pyoil, 35 wt % gaseous products, and 11 wt % solids.

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, methods, and/or steps described in the specification. As one of the ordinary skill in the art will readily appreciate from the disclosure of the present invention, processes, machines, means, methods, and/or steps, presently existing or later to be developed that perform substantially the same function or achieve substantially the same result as the corresponding embodiments described herein, may be utilized according to the present invention. Accordingly, the appended claims are intended to include within their scope such processes, machines, means, methods, and/or steps.

What is claimed is:

1. A catalyst system for depolymerizing polymers, the catalyst system comprising:
a) a zeolite catalyst component; and
b) a co-catalyst, comprising an activated clay component, a solid base component, or a combination thereof.

2. The catalyst system of claim 1, wherein the zeolite catalyst component is one or more members selected from the group consisting of ZSM-5 zeolites, Beta zeolites, Y zeolites, and ultra-stable Y zeolites.

3. The catalyst system of claim 1, wherein the zeolite catalyst component has a SiO₂/Al₂O₃ mole ratio in the range of from 200:1 to 1:1.

4. The catalyst system of claim 1, wherein the activated clay component is one or more members selected from the group consisting of montmorillonite, sauconite, nontronite, hectorite, beidellite, saponite, bentonite, or a combination thereof.

5. The catalyst system of claim 1, wherein the solid base component is one or more members selected from the group consisting of:
a) a layered double hydroxide composition; and
b) an activated carbon composition.

6. The catalyst composition of claim 5, additionally comprising an MR composition, wherein M is an alkali metal, an alkaline earth metal, a transition metal, a post-transition metal, metalloid, or a combination thereof, and R is an oxide, a hydroxide, a carbonate, a sulfate, a sulfide, a nitrate, a nitride, a phosphate, a phosphite, a halide, or a combination thereof.

7. A process for depolymerizing polymers, the process comprising:
a) adding a polyolefin-based feed stream and a first co-catalyst to a first pyrolysis reaction zone to form a first reaction mixture, wherein the first co-catalyst comprises an activated clay component, a solid base component, or a combination thereof;
b) reacting the first reaction mixture under first depolymerization conditions in the absence of oxygen to form a first vapor stream and first liquid stream comprising char;
c) adding the first vapor stream to a first condensation zone, wherein the first vapor stream is subjected to condensing conditions to form a second vapor stream and a second liquid stream;
d) adding the second liquid stream and a zeolite catalyst component to a second pyrolysis reaction zone to form a second reaction mixture;
e) reacting the second reaction mixture under second depolymerization conditions in the absence of oxygen to form a third vapor stream and a third liquid stream comprising char; and
f) adding the third vapor stream to a second condensation zone, wherein the third vapor stream is subjected to condensing conditions to form a fourth vapor stream and a fourth liquid stream, wherein the second liquid product comprises one or more olefin monomers.

8. The process of claim 7, wherein the polyolefin feed stream comprises polyethylene, polypropylene, or a combination thereof.

9. The process of claim 7, wherein the polyolefin feed stream comprises up to 20 wt % of an impurity, wherein the weight percentage is based on the total weight of the polyolefin feed stream.

10. The process of claim 9, wherein the impurity comprises one or more members of the group consisting of polyethylene terephthalate, polystyrene, water, chlorine, or a combination thereof.

11. The process of claim 7, further comprising adding a second co-catalyst to the second pyrolysis reaction zone, wherein the second co-catalyst comprises an activated clay component, a solid base component, or a combination thereof, and the second co-catalyst composition is different than the first co-catalyst composition.

12. The process of claim 7, wherein the first co-catalyst comprises a solid base component.

13. The process of claim 7, wherein the first co-catalyst comprises an activated clay component.

14. The process of claim 11, wherein the second co-catalyst comprises a solid base component.

15. The process of claim 11, wherein the second co-catalyst comprises an activated clay component.

16. The process of claim 7, further comprising adding the second vapor product to the to the second condensation zone.

17. The process of claim 7, wherein the first depolymerization conditions comprise a temperature in the range of from 400° C. to 600° C., a pressure in the range of from 1.0 barg (100 kPa) to 7.0 barg (700 kPa), or a combination thereof.

18. The process of claim 7, wherein the second depolymerization conditions comprise a temperature in the range of from 250° C. to 450° C., a pressure in the range of from 1.0 barg (100 kPa) to 7.0 barg (700 kPa), or a combination thereof.

19. The process of claim 7, wherein the first condensing conditions comprise a temperature in the range of from 20° C. to 250° C., a pressure in the range of from 1.0 barg (100 kPa) to 2.0 barg (200 kPa), or a combination thereof.

20. The process of claim 7, wherein the second condensing conditions independently comprise a temperature in the range of from 20° C. to 100° C., a pressure in the range of from 0.3 barg (30 kPa) to 2.0 barg (200 kPa), or a combination thereof.
